(19) United States
(12) Patent Application Publication
(10) Pub. No.: US 2012/0082726 A1
(43) Pub. Date: Apr. 5, 2012

(54) SURFACE-TREATED MODAFINIL PARTICLES

(75) Inventors: Salah U. AHMED, New City, NY (US); Muhammed A. HOSSAIN, Congers, NY (US); Pruthvi R. KATIKANENI, Boonton, NJ (US); Zhijun JIANG, Kinnelon, NJ (US)

(73) Assignee: CEPHALON, INC., Frazer, PA (US)

(21) Appl. No.: 13/324,529

(22) Filed: Dec. 13, 2011

Related U.S. Application Data
(63) Continuation of application No. 12/683,231, filed on Jan. 6, 2010, now abandoned.
Application No. 12/683,231 is a continuation of application No. 10/834,390, filed on Apr. 29, 2004, now abandoned.
(60) Provisional application No. 60/466,045, filed on Apr. 29, 2003.

(51) Int. Cl. A61K 31/165; A61P 25/00; A61P 25/28; A61K 9/14; A61P 25/16

(52) U.S. Cl. 424/489; 514/618

(57) ABSTRACT

The present invention is directed to solid oral dosage forms comprising surface-treated particles comprising modafinil particles and a hydrophilic treating agent, methods of making the same, and uses thereof.

This application claims priority to U.S. Provisional Patent Application 60/466,045, filed Apr. 29, 2003, which is incorporated herein in its entirety.

### BACKGROUND OF THE INVENTION

1. Field of the Invention

The present invention is directed to solid oral dosage forms comprising surface-treated particles comprising modafinil particles and a hydrophilic treating agent, methods of making the same, and uses thereof.

2. Background Art

Modafinil, Formula I, is an acetamide derivative and is also known as 2-(benzhydrylsulfinyl)acetamide, or 2-[(diphenylmethyl)sulfinyl)]acetamide.

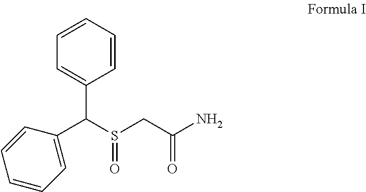

Modafinil exerts a wakefulness-promoting effect on humans and animals. A single administration of modafinil results in increased locomotor activity in mice and increased nocturnal activity in monkeys (Duteil et al., Eur. J. Pharmacol. 180: 49 (1990)). Human testing demonstrated the use of modafinil for treatment of idiopathic hypersomnia and narcolepsy (Basuji et al., Prog. Neuro-Psych. Biol. Psych. 12: 695 (1988)). According to the United States Food and Drug Administration, modafinil racemate is approved for treatment of narcolepsy.

Other uses of modafinil are known in the art. Some of the disclosed uses of modafinil are in treatment of Parkinson's disease (U.S. Pat. No. 5,180,745), as an anti-ischemic agent (European Published Application 547952), and for treatment of urinary incontinence (European Published Application 594507). U.S. Pat. No. 4,927,855 discloses the levorotatory form of modafinil and the use of the levorotatory form for treating hypersomnia and Alzheimer's disease.

Previous pharmaceutical preparations of modafinil have been, described: U.S. Pat. Nos. 4,177,290; 5,180,745; 5,618,845; 6,489,363; RE37,516; and U.S. Published Application Nos. 2002/0098240 and 2002/0160982.

It has, been observed that modafinil exhibits poor water and lipid solubility, and it is therefore difficult to solubilize modafinil in pharmaceutically-acceptable compositions. A need exists in the art for modafinil formulations that are readily absorbed and have a reliable dosing profile.

### BRIEF SUMMARY OF THE INVENTION

It is an object of the invention to provide a solid oral dosage form comprising surface-treated particles comprising modafinil particles treated with a hydrophilic treating agent, methods of making the same, and uses thereof.

It is an object of the invention to provide: a solid oral dosage form comprising surface-treated particles comprising (a) modafinil particles, wherein greater than 5% of cumulative total of pretreated modafinil particles have a diameter of 220 μm or greater, and the pretreated modafinil particles have a median diameter of 70 μm to about 200 μm, and (b) a hydrophilic treating agent; wherein the modafinil particles of (a) are surface-treated with the hydrophilic treating agent of (b).

It is an object of the invention to provide a solid oral dosage form comprising surface-treated particles comprising (a) modafinil particles, wherein greater than 5% of cumulative total of pretreated modafinil particles have a diameter of 220 μm or greater, and the pretreated modafinil particles have a mean diameter of about 30 μm to about 200 μm, and (b) a hydrophilic treating agent; wherein the modafinil particles of (a) are surface-treated with the hydrophilic treating agent of (b).

It is an object of the present invention to provide a method of making a solid oral dosage form, the method comprising (a) combining modafinil particles and a hydrophilic treating agent to generate granules of surface-treated modafinil particles, wherein greater than 5% of cumulative total of pretreated modafinil particles have a diameter of 220 μm or greater, and the pretreated modafinil particles have a median diameter of 70 μm to about 200 μm, and (b) forming the granules of (a) into a solid oral dosage form.

It is an object of the present invention to provide a method of making a solid oral dosage form, the method comprising (a) combining modafinil particles and a hydrophilic treating agent to generate granules of surface-treated modafinil particles, wherein greater than 5% of cumulative total of pretreated modafinil particles have a diameter of 220 μm or greater, and the pretreated modafinil particles have a mean diameter of about 30 μm to about 200 μm, and (b) forming the granules of (a) into a solid oral dosage form.

The present invention is also directed to the solid oral dosage form made by the method of the invention as described herein, and uses thereof.

### BRIEF DESCRIPTION OF THE FIGURES

FIG. 1. Dissolution profiles of tablets from pretreated (A) and surface-treated (B) modafinil particles. Both the pretreated and surface-treated modafinil particles originated from the same production lot (median particle size 148 μm; 5% of the total of pretreated modafinil particles have a diameter of greater than 277 μm).

### DETAILED DESCRIPTION OF THE INVENTION

The present invention provides surface treatment of particles with a hydrophilic treating agent, wherein the surface-treated particles can be more readily absorbed by the body when compared to pretreated or non-treated modafinil particles. Additionally, due to the ready absorption of the surface-treated particles of the present invention, the safety profile of the drug can be more accurately controlled.

The present invention is directed to a solid oral dosage form comprising surface-treated particles comprising (a) modafinil particles, wherein greater than 5% of cumulative total of pretreated modafinil particles have a diameter of 220 μm or greater, and the pretreated modafinil particles have a median diameter of 70 μm to about 200 μm, and (b) a hydrophilic treating agent, wherein the modafinil particles of (a) are surface-treated with the hydrophilic treating agent of (b).

The present invention is directed to a solid oral dosage form comprising surface-treated particles comprising (a) modafinil particles, wherein greater than 5% of cumulative total of pretreated modafinil particles have a diameter of 220 μm or greater, and the pretreated modafinil particles have a mean diameter of about 30 μm to about 200 μm, and (b) a hydrophilic treating agent, wherein the modafinil particles of (a) are surface-treated with the hydrophilic treating agent of (b).

The present invention is directed to a method of making a solid oral dosage form, the method comprising (a) combining modafinil particles and a hydrophilic treating agent to generate granules of surface-treated modafinil particles, wherein greater than 5% of cumulative total of pretreated modafinil particles have a diameter of 220 μm or greater, and the pretreated modafinil particles have a median diameter of 70 μm to about 200 μm, and (b) forming the granules of (a) into a solid oral dosage form.

The present invention is directed to a method of making a solid oral dosage form, the method comprising (a) combining modafinil particles and a hydrophilic treating agent to generate granules of surface-treated modafinil particles, wherein greater than 5% of cumulative total of pretreated modafinil particles have a diameter of 220 μm or greater, and the pretreated modafinil particles have a mean diameter of about 30 μm to about 200 μm, and (b) forming the granules of (a) into a solid oral dosage form.

In the methods of the invention, the modafinil particles of (a) are combined with a diluent and a disintegrant prior to combining with the hydrophilic treating agent. In some embodiments, the granules of (a) can be dried. In some embodiments, the granules of (a) can be milled. In some embodiments, the granules of (a) can be combined with an excipient and a lubricant to form a blend of surface-treated modafinil particles. In some embodiments, the solid oral dosage form of (b) can be formed by compressing or encapsulating the granules of (a).

As used herein, “solid oral dosage form” means a dose which can be administered to a patient, and which can be readily handled and packaged, remaining as a physically and chemically stable unit dose, comprising either a modafinil compound or a pharmaceutically acceptable composition comprising a modafinil compound. A solid oral dosage form includes, but is not limited to, a tablet, coated tablet, caplet, coated caplet, dragee, or capsule. In some embodiments, the solid oral dosage form can be a tablet or a capsule. As described herein, the solid oral dosage form comprises modafinil particles, and therefore the modafinil contained in the solid oral dosage form is not in solution. The amount of modafinil in the solid oral dosage form can vary. For example, the solid oral dosage form can include 100 mg, 200 mg, 300 mg or 400 mg of modafinil.

The present invention is also directed to the solid oral dosage form made by the method of the invention as described herein.

The solid oral dosage form of the present invention can comprise modafinil particles wherein greater than 5% of cumulative total of pretreated particles have a diameter of 220 μm or greater. In some embodiments, the solid oral dosage form of the present invention can comprise modafinil particles wherein greater than 6% of cumulative total of pretreated particles have a diameter of 220 μm or greater. In some embodiments, the solid oral dosage form of the present invention can comprise modafinil particles wherein greater than 10% of cumulative total of pretreated particles have a diameter of 220 μm or greater. In some embodiments, the solid oral dosage form of the present invention can comprise modafinil particles wherein greater than 15% of cumulative total of pretreated particles have a diameter of 220 μm or greater. In some embodiments, the solid oral dosage form of the present invention can comprise modafinil particles wherein greater than 20% of cumulative total of pretreated particles have a diameter of 220 μm or greater. In some embodiments, the solid oral dosage form of the present invention can comprise modafinil particles wherein greater than 30% of cumulative total of pretreated particles have a diameter of 220 μm or greater.

The solid oral dosage form of the present invention can comprise pretreated modafinil particles having a median diameter of 70 μm to about 200 μm. In some embodiments, the solid oral dosage form of the present invention can comprise pretreated modafinil particles having a median diameter of about 80 μm to about 170 μm. In some embodiments, the solid oral dosage form of the present invention can comprise pretreated modafinil particles having a median diameter of about 90 μm to about 150 μm.

The solid oral dosage form of the present invention can comprise pretreated modafinil particles having a mean diameter of about 30 μm to about 200 μm. In some embodiments, the solid oral dosage form of the present invention can comprise pretreated modafinil particles having a mean diameter of about 40 μm to about 170 μm. In some embodiments, the solid oral dosage form of the present invention can comprise pretreated modafinil particles having a mean diameter of about 50 μm to about 150 μm.

The solid oral dosage form of the present invention can comprise pretreated modafinil particles wherein at least 95% of cumulative total of the pretreated modafinil particles have a diameter of less than about 500 μm. In some embodiments, the solid oral dosage form of the present invention can comprise pretreated modafinil particles wherein at least 95% of cumulative total of the pretreated modafinil particles have a diameter of less than about 400 μm. In some embodiments, the solid oral dosage form of the present invention can comprise pretreated modafinil particles wherein at least 95% of cumulative total of the pretreated modafinil particles have a diameter of less than about 350 μm.

As used herein, “modafinil” refers to modafinil or modafinil compounds, its racemic mixtures, substantially individual enantiomers, acid addition salts, such as metabolic acids of modafinil, benzhydrylsulfinylacetic acids, and its sulfone forms, hydroxylated forms, polymorphic forms, analogs, derivatives, cogeners, prodrugs, and compounds made by mixtures thereof. Prodrugs are known in the art as compounds that are converted to the active agent (a modafinil compound) in the body of a subject. These and other modafinil compounds, and their preparation, have been disclosed in U.S. Pat. Nos. 4,177,290; 4,927,855; 5,719,168; and U.S. Patent Application No. 60/204,789.

As used herein, “modafinil particles” refer to aggregated physical units of modafinil, i.e., pieces, granules, or grains of modafinil.

“Pretreated modafinil particles” refer to modafinil particles that have not been mixed, treated, sprayed, or encountered any other form of contact with a hydrophilic treating agent.

As used herein, “surface” refers to the periphery or the outer boundary of the particle, independent of the shape of the particle. “Surface-treated particles” refer to particles that have been mixed, treated, sprayed, or encountered any other form of contact with a hydrophilic treating agent. Surface-treated particles include particles wherein the hydrophilic treating agent remains on the surface of the particle and/or is adsorbed or absorbed into the particles. Surface treatment of particles with a hydrophilic treating agent can improve wettability that in turns facilitates dissolution and in vivo absorption in a subject.

Optionally, the modafinil particles are a racemic mixture. The term “racemic mixture” refers to a mixture that is composed of approximately equal amounts of dextrorotatory (+) and levorotatory (−) forms of modafinil and is not significantly optically active. Optionally the modafinil particles can be of unequal amounts of dextrorotatory (+) and levorotatory (−) forms of modafinil. Optionally, the modafinil particles can be a dextrorotatory (+) or levorotatory (−) enantiomer, or an excess of one over the other.

As used herein, “about” refers to plus or minus 10% of the indicated number. For example, “about 200 μm” indicates a range of 180 μm to 220 μm; “about 10%” indicates a range of 9% to 11%.

Methods for preparing modafinil appear in U.S. Pat. No. 4,177,290 (hereinafter the '290 patent). Modafinil of the particle size defined herein can be obtained by a variety of approaches utilizing conventional methods (O'Conner et al., Ch. 88, Remington's Pharmaceutical Sciences, 18^{th} Edition, Mack Publishing Co., Easton, Pa. (1990)).

The term “particle size” refers to the particle diameter. As used herein, the term “diameter” is a volumetric measurement based on the presumed spherical shape of modafinil particles.

As used herein, the term “mean diameter,” when used in reference to the size of modafinil particles, refers to the sum of the diameter measurements of all measurable particles measured, divided by the total number of particles measured. For example, for five measurable particles determined to have diameters of 230 μm, 200 μm, 350 μm, 200 μm, and 200 μm, the mean diameter would be 236 μm.

As used herein, the term “median diameter,” when used in reference to the size of modafinil particles, indicates that about 50% of all measurable particles measured have a particle diameter less than the defined median particle diameter value, and that about 50% of all measurable particles measured have a particle diameter greater than the defined median particle diameter value. For example, for five measurable particles determined to have diameters of 200 μm, 210 μm, 250 μm, 270 μm and 300 μm, the median diameter would be 250 μm.

Various hydrophilic treating agents can be used in the invention. The term “hydrophilic treating agent” refers to an agent that improves the association of a particle with water. Hydrophilic treating agents include, but are not limited to, polyvinylpyrrolidones, polyethylene glycols, polyols, microcrystalline celluloses, hydroxypropyl celluloses, hydroxypropyl methyl celluloses, methyl celluloses, sorbitols, monosaccharides, disaccharides, polysaccharides, starches, sugars and sugar derivatives, lactoses, crospovidones, hydrophilic carbohydrates, and combinations thereof. In some embodiments, the hydrophilic treating agent can include polyvinylpyrrolidones, lactoses, crospovidones, and combinations thereof. The hydrophilic treating agent can be in any physical state, including, but not limited to, a liquid, a solution, a suspension, a solid, or an aerosol.

The present invention can further comprise a pharmaceutically acceptable inactive agent. As used herein, “inactive agent” refers to a substance that is used in the formulation of pharmaceutical compositions, and, by itself, generally has little or no therapeutic value. Various inactive agents can be used in the invention. As used herein, the term “pharmaceutically acceptable” inactive agent refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. In some embodiments, the pharmaceutically acceptable inactive agent can be a diluent, a disintegrant, an excipient, a lubricant, or combinations thereof. Inactive agents include, but are not limited to, diluents, disintegrants, lubricants, excipients, antioxidants, anti-bacterial agents and other preservatives, chelating agents, buffering agents, agents for adjusting toxicity, colorings, flavorings and diluting agents, emulsifying and suspending agents, and other substances with pharmaceutical applications.

Various diluents can be used in the invention. A diluent can be considered as any inert substance, or mixture of substances, added to increase the bulk of the pharmaceutical formulation in order to make the solid oral dosage form a practical size for administration or compression. Diluents include, but are not limited to, lactose (e.g., anhydrous and/or monohydrate), mannitol, xylitol, microcrystalline cellulose, sugar, dextrin, hydrophilic carbohydrate, and combinations thereof. In some embodiments, the diluent can be lactose.

Various disintegrants can be used in the invention. A disintegrant can be considered as any substance, or mixture of substances, added to a solid oral dosage form to facilitate its breakup or disintegration after administration. Disintegrants include, but are not limited to, crospovidone, croscarmellose sodium, polacrilin potassium, sodium starch glycolate, starch, and combinations thereof. In some embodiments, the disintegrant can be crospovidone.

Various excipients can be used in the invention. An excipient can be considered as any substance, or mixture of substances, added to a solid oral dosage form to give additional desirable physical characteristics to the formulation. Excipients include, but are not limited to, lactose, colloidal silicon dioxide, fumed silicon dioxide, mannitol, xylitol, microcrystalline cellulose, sugar, dextrin, hydrophilic carbohydrate, and combinations thereof. In some embodiments, the excipient can comprise lactose and colloidal silicon dioxide.

Various lubricants can be used in the invention. A lubricant can be considered as any substance, or mixture of substances, that can prevent adhesion of the solid oral dosage form material to the surface of the dies and punches, reduce interparticle friction, facilitate the ejection of the solid oral dosage from from the die cavity, and/or improve the rate of flow of the solid oral dosage from particles. Lubricants include, but are not limited to, sodium stearyl fumarate, talc, magnesium stearate, stearic acid, hydrogenated vegetable oil, and combinations thereof. In some embodiments, the lubricant can comprise sodium stearyl fumarate and talc.

As mentioned above, a hydrophilic treating agent includes, but is not limited to, polyvinylpyrrolidone, a synthetic polymer consisting essentially of linear 1-vinyl-2-pyrrolidinone groups of the following repeating structural motif:

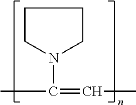

### See, e.g., The U.S. Pharmacopeia & The National Formulary, USP 23-NF 18 (1995), p. 1267-1268.

Crospovidone is a water-insoluble synthetic cross-linked homopolymer of N-vinyl-2-pyrrolidone. Crospovidone acts as a disintegrant and dissolution aid for tablets, capsules, and pellets. See, e.g., The U.S. Pharmacopeia & The National Formulary, USP 23-NF 18 (1995), pp. 2239.

A hydrophilic carbohydrate is any carbohydrate that can form a true or colloidal suspension in water. Carbohydrates are polyhydroxy aldehydes, polyhydroxy ketones, or compounds that can be hydrolyzed to form them. Examples of hydrophilic carbohydrates include, but are not limited to, a carboxymethyl cellulose, a methyl cellulose gum, a propyl cellulose, a methyl cellulose sugar, a sucrose or a sucrose derivative, a xylose, and a lactose.

A sugar derivative includes, but is not limited to, the alcohol, acid, ketone, aldehyde, and amino forms of the sugar.

Percentage weight of each component in the solid oral dosage form can vary. Modafinil particles can be about 10% to about 80% by weight of the dosage form, about 20% to about 65% by weight of the dosage form, or about 30% to about 50% by weight of the dosage form. Optionally, modafinil particles can be about 40% by weight of the dosage form. Polyvinylpyrrolidone can be about 0.1% to about 20% by weight of the dosage form, about 2% to about 10% by weight of the dosage form, or about 3% to about 5% by weight of the dosage form. Optionally, polyvinylpyrrolidone can be about 4% by weight of the dosage form. Lactose used as a diluent can be about 5% to about 60% or about 10% to about 40% by weight of the dosage form. Optionally, lactose used as a diluent can be about 20% by weight of the dosage form. Crospovidone can be about 3% to about 50% by weight of the dosage form, about 4% to about 25% by weight of the dosage form, or about 8% to about 16% by weight of the dosage form. Optionally, crospovidone can be about 12% by weight of the dosage form. Lactose used as an excipient can be about 5% to about 60% or about 10% to about 40% by weight of the dosage form. Optionally, lactose used as an excipient can be about 22% by weight of the dosage form. Colloidal silicon dioxide can be about 0.01% to about 5% by weight of the dosage form, about 0.01% to about 2% by weight of the dosage form, or about 0.01% to about 1% by weight of the dosage form. Optionally, colloidal silicon dioxide can be about 0.4% by weight of the dosage form. Sodium stearyl fumarate can be about 0.01% to about 5% by weight of the dosage form, about 0.1% to about 2% by weight of the dosage form, or about 0.5% to about 1.5% by weight of the dosage form. Optionally, sodium stearyl fumarate can be about 1% by weight of the dosage form. Talc can be about 0.01% to about 10% by weight of the dosage form, about 0.1% to about 5% by weight of the dosage form, or about 0.5% to about 2% by weight of the dosage form. Optionally, talc is about 1% by weight of the dosage form.

Any suitable drying temperature can be used to dry the modafinil particles treated with the hydrophilic treating agent. For example, the drying temperature of about 20° C. to about 80° C. can be used to dry the modafinil particles surface-treated with the hydrophilic treating agent. Optionally, a temperature of about 40° C. to about 70° C. can be used to dry the modafinil particles surface-treated with the hydrophilic treating agent. Optionally, a temperature of about 50° C. to about 60° C. can be used to dry the modafinil particles surface-treated with the hydrophilic treating agent. Optionally, the drying temperature can be about 55° C.

In one aspect, the present invention is directed to a solid oral dosage form comprising surface-treated particles comprising (a) modafinil particles, wherein greater than 5% of cumulative total of pretreated modafinil particles have a diameter of 220 μm or greater, at least 95% of cumulative total of pretreated modafinil particles have a diameter of less than about 400 μm, the pretreated modafinil particles have a median diameter of 70 μm to about 200 μm, the pretreated modafinil particles have a mean diameter of about 30 μm to about 200 μm, and the modafinil particles are about 30% to about 50% by weight of the dosage form, (b) a hydrophilic treating agent comprising a polyvinylpyrrolidone, wherein the polyvinylpyrrolidone is about 3% to about 5% by weight of the dosage form, (c) a diluent comprising lactose, wherein the lactose is about 10% to about 40% by weight of the dosage form, (d) disintegrant comprising crospovidone, wherein the crospovidone is about 8% to about 16% by weight of the dosage form, (e) an excipient comprising colloidal silicon dioxide and lactose, wherein the colloidal silicon dioxide is about 0.01% to about 1% by weight of the dosage form and lactose is about 10% to about 40% by weight of the dosage form, and (f) a lubricant comprising sodium stearyl fumarate and talc, wherein the sodium stearyl fumarate is about 0.5% to about 1.5% by weight of the dosage form, and the talc is about 0.5% to about 2% by weight of the dosage form, wherein the modafinil particles of (a) are surface-treated with the hydrophilic treating agent of (b).

In another aspect, the present invention is directed to a method of making a solid oral dosage form, the method comprising (a) mixing modafinil particles with a diluent and a disintegrant to generate a mixture, wherein (i) greater than 5% of cumulative total of pretreated modafinil particles have a diameter of 220 μm or greater, (ii) at least 95% of cumulative total of pretreated modafinil particles have a diameter of less than about 400 μm, (iii) the pretreated modafinil particles have a median diameter of 70 μm to about 200 μm, (iv) the pretreated modafinil particles have a mean diameter of about 30 μm to about 200 μm, (v) the modafinil particles are about 30% to about 50% by weight of the dosage form, (vi) the diluent comprises lactose, wherein the lactose is about 10% to about 40% by weight of the dosage form, and (vii) the disintegrant comprises crospovidone, wherein the crospovidone is about 8% to about 16% by weight of the dosage form, (b) adding a hydrophilic treating agent onto the mixture of (a) while mixing the mixture to generate granules of surface-treated modafinil particles, wherein the hydrophilic treating agent comprises a polyvinylpyrrolidone and the polyvinylpyrrolidone is about 3% to about 5% by weight of the dosage form, (c) drying the granules of surface-treated modafinil particles of (b), (d) milling the dried granules of surface-treated modafinil particles of (c), (c) mixing the milled surface-treated modafinil particles of (d) with an excipient and a lubricant to form a dry blend of surface-treated modafinil particles, wherein (i) the excipient comprises colloidal silicon dioxide and lactose, wherein the colloidal silicon dioxide is about 0.01% to about 1% by weight of the dosage form and the lactose is about 10% to about 40% by weight of the dosage form, and (ii) the lubricant comprises sodium stearyl fumarate and talc, wherein the sodium stearyl fumarate is about 0.5% to about 1.5% by weight of the dosage form and the talc is about 0.5% to about 2% by weight of the dosage form, and (f) compressing or encapsulating the dry blend of (e) into a solid oral dosage form.

Particle size can be determined by various methods known to those in the art, such as, but not limited to, laser particle size detectors. Particle size, as defined herein, is determined using a Hiac/Royco system, which includes the Model 9064 particle counter and the Model 3000A liquid syringe sampler (11801 Tech Road, Silver Spring, Md. 20904, U.S.A.).

The solid oral dosage-form of the present invention can be useful in the treatment of, e.g., idiopathic hypersomnia, narcolepsy, or related sleep disorders in a subject or patient. The present invention can also be useful for providing a neuroprotective effect in a subject or patient, e.g., Parkinson's disease. Additionally, the present invention can have potential benefit for the treatment of, e.g., depression, hypersomnia, Alzheimer's disease, anti-ischemic disorders, or urinary incontinence in a subject or patient.

All of the various embodiments or options described herein can be combined in any and all variations.

The following Example serves only to illustrate the invention, and is not to be construed in any way to limit the invention.

### EXAMPLE

Lactose monohydrate, modafinil, and crospovidone were passed though a Russell Finex Compact sieve (Russell Finex, N.C.) fitted with 30-mesh screen (30 openings per linear inch) with the vibrasonic and the shaker on. The screened materials were then mixed in a 300 liter high shear mixer/granulator for three minutes. While mixing the pretreated mixture, a 15% polyvinylpyrrolidone solution was added to the mixture and mixed for five additional minutes to generate granules of surface-treated modafinil particles. The surface-treated modafinil particles were then dried in a fluid bed dryer until the moisture content was not more than 2%. The dried, surface-treated modafinil particles were milled using a Fitz Mill Model D (Fitz-Patrick, Ill.) fitted with a screen mesh with a 0.05 inch opening (e.g., Fitz-Patrick screen #1532-0050) with knives forward at medium speed. The resulting milled particles were then mixed for three minutes with anhydrous lactose and crospovidone, which had previously been passed though a Russell Finex Compact Sieve (Russell Finex, N.C.) fitted with 30-mesh screen with the vibrasonic and shaker on. Finally, colloidal silicon dioxide, sodium stearyl fumarate, and talc, which were previously passed though a Russell Finex Compact sieve fitted with a 30-mesh screen with the vibrasonic and shaker on, were added to the surface-treated mixture for 30 seconds to form a final blend. The final blend was then compressed in a Kikusui Libra tablet press (Kikusui Seisakusho, Ltd., Japan) to form tablets.

Particle size was determined using a Hiac/Royco system, which includes the Model 9064 particle counter and the Model 3000A liquid syringe sampler (11801 Tech Road, Silver Spring, Md. 20904, U.S.A.). Samples were prepared in water saturated with modafinil. For each sample preparation, five consecutive readings were measured. The test was done in triplicate and the average was reported.

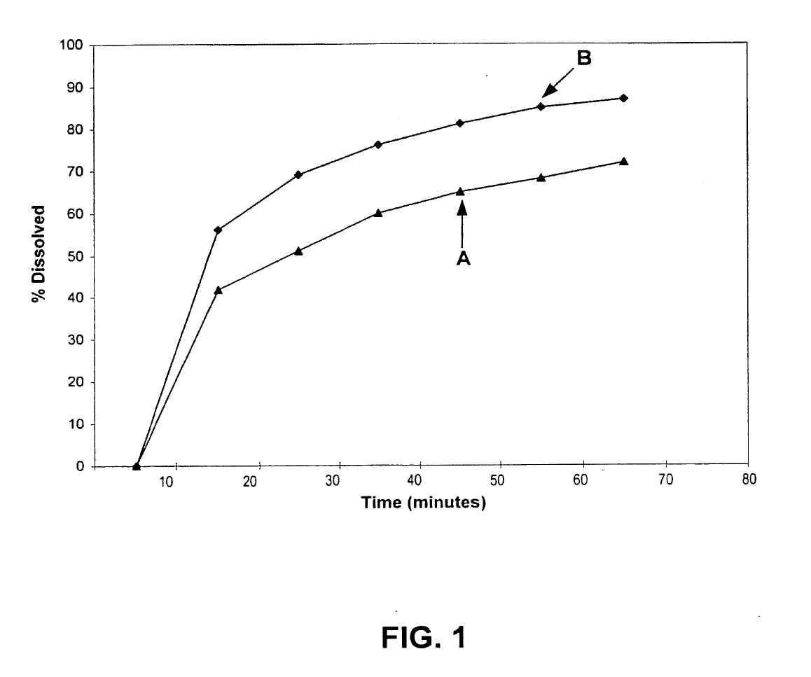

Various methods of determining dissolution profiles are known in the art. Dissolution profiles of tablets from pretreated (A) and surface-treated (B) modafinil particles were examined using a USP Apparatus II-paddle at 50 RPM in 900 mL 0.1N hydrochloride (FIG. 1).

This example illustrates one possible formulation of the present invention. While the invention has been particularly shown and described with reference to some embodiments thereof, it will be understood by those skilled in the art that they have been presented by way of example only, arid not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A method of making a solid oral dosage form, said method comprising:
(a) combining dry modafinil particles with a solution of a hydrophilic treating agent to generate granules of surface-treated modafinil particles, wherein greater than 5% of cumulative total of pretreated modafinil particles have a diameter of 220 μm or greater, and the pretreated modafinil particles have a median diameter of 70 μm to about 200 μm; and
(b) forming the granules of (a) into a solid oral dosage form.

2. The method of claim 1, wherein the dry modafinil particles of (a) are combined with a diluent and a disintegrant prior to combining with the solution of a hydrophilic treating agent.

3. The method of claim 1, wherein the granules of (a) are dried prior to step (b).

4. The method of claim 3, wherein the dried granules are milled prior to step (b).

5. The method of claim 1, wherein the granules of (a) are combined with an excipient and a lubricant to form a blend of surface-treated modafinil particles.

6. The method of claim 1, wherein the pretreated modafinil particles have a mean diameter of about 30 μm to about 200 μm.

7. The method of claim 1, wherein at least 95% of cumulative total of the pretreated modafinil particles have a diameter of less than about 400 μm.

8. The method of claim 1, wherein the hydrophilic treating agent is selected from the group consisting of a polyvinylpyrrolidone, a polyethylene glycol, a polyol, a microcrystalline cellulose, a hydroxypropyl cellulose, a hydroxypropyl methyl cellulose, a methyl cellulose, a sorbitol, a monosaccharide, a disaccharide, a polysaccharide, a starch, a lactose, a crospovidone, a hydrophilic carbohydrate, and combinations thereof.

9. The method of claim 1, wherein the hydrophilic treating agent is selected from the group consisting of a polyvinylpyrrolidone, a lactose, a crospovidone, and combinations thereof.

10. The method of claim 1, wherein the hydrophilic treating agent is a polyvinylpyrrolidone.

11. The method of claim 1, wherein the modafinil particles are a racemic mixture of modafinil.

12. The method of claim 2, wherein the diluent is selected from the group consisting of a lactose, a mannitol, a xylitol, a microcrystalline cellulose, a sugar, a dextrin, a hydrophilic carbohydrate, and combinations thereof.

13. The method of claim 2, wherein the diluent is lactose.

14. The method of claim 2, wherein the disintegrant is selected from the group consisting of a crospovidone, a croscarmellose sodium, a polacrilin potassium, a sodium starch glycolate, a starch, and combinations thereof.

15. The method of claim 2, wherein the disintegrant is crospovidone.

16. The method of claim 5, wherein the excipient is selected from the group consisting of a lactose, a colloidal silicon dioxide, a fumed silicon dioxide, a mannitol, a xylitol, a microcrystalline cellulose, a sugar, a dextrin, a hydrophilic carbohydrate, and combinations thereof.

17. The method of claim 5, wherein the excipient is a combination of lactose and colloidal silicon dioxide.

18. The method of claim 5, wherein the lubricant is selected from the group consisting of a sodium stearyl fumarate, a talc, a magnesium stearate, a stearic acid, a hydrogenated vegetable oil, and combinations thereof.

19. The method of claim 5, wherein the lubricant is a combination of sodium stearyl fumarate and talc.

20. The method of claim 3, wherein the granules are dried at a temperature of about 40° C. to about 80° C.
